(19) United States
(12) Patent Application Publication
(10) Pub. No.: US 2006/0269928 A1
(43) Pub. Date: Nov. 30, 2006

(54) Compositions, methods and apparatus for supercritical fluid virus inactivation

(75) Inventor: Trevor Percival Castor, Arlington, MA (US)

Correspondence Address: Aphios Corporation, 3-E Gill Street, Woburn, MA, 01801, US

(73) Assignee: Aphios Corporation

(21) Appl. No.: 11/139,836

(22) Filed: May 27, 2005

(51) Int. Cl. C12Q 1/70; C12Q 1/68

(52) U.S. Cl. 435/6; 435/5

(57) ABSTRACT

The present invention is directed to a composition of critical, supercritical or near critical fluid and apparatus for inactivating viruses associated or potentially associated with protein derived samples and methods of their use.

### RELATED APPLICATION AND PATENTS

This application claims priority to U.S. provisional application for patent U.S. Ser. No. 60/574,696, filed May 26, 2004.

### FEDERALLY FUNDED RESEARCH

Research leading to this application was in part funded by the National Institute of Standards and Testing, United States Department of Commerce under Cooperative Agreement No. 70NANB2H1256.

### FIELD OF INVENTION

The present invention relates generally to the inactivation of viruses in protein-derived products from blood, cells, microorganisms and recombinant DNA technology. In particular, the instant invention pertains to compositions, methods and apparatus for inactivating viruses in protein derived products.

### BACKGROUND OF INVENTION

Viral transmission of HIV, hepatitis A and B through blood and plasma products has lead to increased donor screening and application of viral inactivation techniques in the manufacture of blood products. While screening has contributed significantly to reducing the risk, the risk for individual blood components remains too high. Current techniques for viral inactivation are insufficient, given their variability in inactivating certain enveloped viruses such as hepatitis C, and their inability to inactivate non-enveloped viruses such as hepatitis A and parvoviruses. For example, there have been several recent European reports of hepatitis A transmission to recipients of solvent/detergent treated Factor VIII concentrate.

This invention utilizes critical, supercritical or near-critical fluids for the gentle and rapid inactivation of both enveloped and non-enveloped viruses without any significant alteration of product quality and biological activity. This application will use the term SCoNCF to represent a supercritical, critical or near critical fluid with or without polar cosolvents. This application incorporates by reference the definition of terms set forth in U.S. Pat. No. 5,877,005.

A critical fluid of interest is gas at its critical temperature and critical pressure. A supercritical fluid of interest is a gas at or above its critical pressure or/and at or above its critical temperature. For the purpose of this discussion, there is no distinction to be made between a critical and supercritical fluid. These fluids are gases at ambient temperature and pressure conditions. As shown in FIG. 1, a pure component compound enters its supercritical fluid region at conditions, which equal or exceed both its critical temperature and critical pressure. These parameters are intrinsic thermodynamic properties of all pure component compounds. Carbon dioxide, for example, becomes supercritical at conditions equal to or exceeding 31.1° C. and 72.8 atm. In these supercritical or near-critical fluid regions, normally gaseous substances such as carbon dioxide become dense phase fluids, which exhibit greatly enhanced solvating power. At a pressure of 204 atm, and a temperature of 40° C., carbon dioxide behaves much like an organic solvent. The term “near critical fluid” is used to refer to a fluid that is below its critical temperature and/or pressure but has density or solvating properties of a critical fluid. Polar cosolvents are fluids such as methanol and ethanol that are used in molar ratios less than 50% but typically about 5%.

This application uses the term “protein rich” or “protein derived” to mean samples and solutions that have as a major component, proteins. Protein rich materials are used in medicine, foodstuffs and cosmetics. For example, without limitation, treated fetal bovine serum, human plasma proteins such as Factor VIII and immunoglobulins, collagen, sensitive natural enzymes such as alkaline phosphatase and α₁-protease inhibitor and recombinant proteins such as biosynthetic insulin.

### SUMMARY OF THE INVENTION

Four fundamental steps are required for SCoNCF critical fluid viral inactivation (CFI). SCoNCF must be first added to the product, which must then be brought to the appropriate pressure and temperature conditions. Next, the aqueous sample must be mixed with SCoNCF. Finally, the sample must be decompressed to ambient pressure. The mixing step is an area, which is of paramount importance in the design and engineering continuous flow CFI equipment. The mixing step is very important since most SCoNCF and proteinaceous solutions are relatively immiscible with each other. Mixing will affect the efficiency with which virus particles are contacted with the SCoNCF and their subsequent inactivation. Efficient mixing will also reduce processing time, improve manufacturing throughput per unit of capital equipment and significantly reduce overall manufacturing costs.

There are several types of mixing, which are traditionally carried out for immiscible and partly immiscible fluids. Most of these types fall into the category of turbulent mixing devices such as the Continuous Stirred Tank Reactor (CSTR) shown as FIG. 2 and static in-line mixers used in our previous patent application. Turbulent mixing is defined as the regime where the Reynolds Number, which is the ratio of inertia to viscous forces, is equal to or greater than 2,000. We have found that approximately 30 minutes to 2 hours of mixing are required for efficient viral inactivation using SCoNCF with turbulent flow mixing; other disadvantages may include some protein denaturation especially with shear-sensitive materials.

We have discovered that viral inactivation time can be significantly reduced and protein loss minimized by diffusing the SCoNCF into laminar, small-diameter aqueous droplets or streams. The basic concept is to inject an aqueous droplet or stream into an isobaric mixing chamber containing the SCoNCF as shown in FIG. 3. Laminar flow conditions are maintained in the sample by choosing the flow rate low enough to obtain Reynolds numbers less than 2,000, i.e. below the turbulent transition number. Time required to approach the equilibrium concentration of SCoNCF by diffusion into the aqueous droplet or stream can be tailored by choosing the injector inner diameter, length of the mixing section, and flow rate. This approach confers several advantages: (1) shear forces are minimized, reducing possible damage to proteins; (2) contact of the aqueous stream with the walls of the mixer can be minimized, reducing possible protein damage; and (3) mixing geometry is simple, amenable to mathematical analysis, and scalable. Volume throughput can be scaled by increasing the cross-sectional area of the isobaric mixing chamber; inactivation can be increased by adding stages as shown in FIG. 4.

### BRIEF DESCRIPTION OF THE DRAWINGS

FIG. 1 is a supercritical fluid diagram.

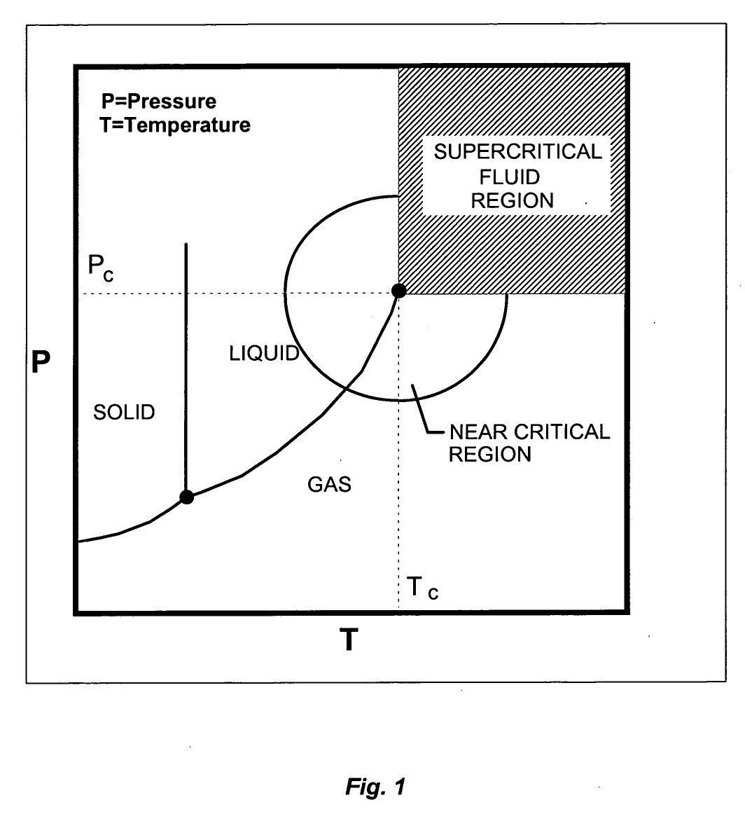

FIG. 2 is a turbulent mixing SCoNCF CFI unit.

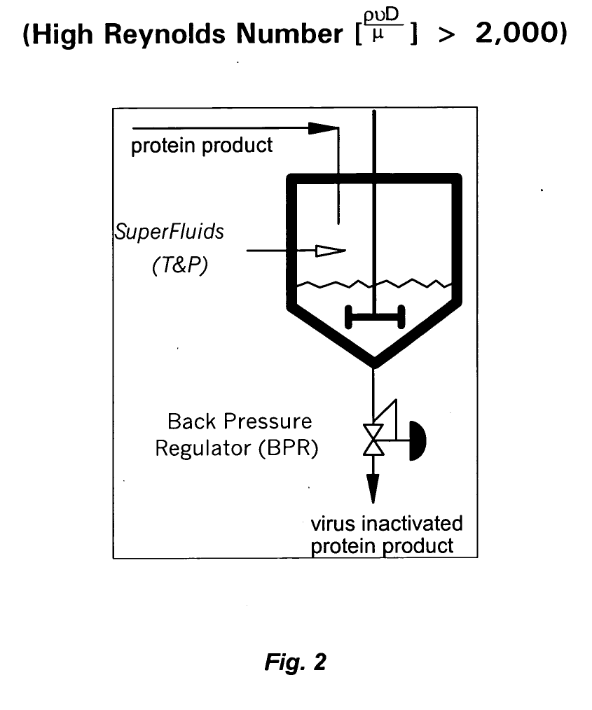

FIG. 3 is a laminar flow SCoNCF CFI unit.

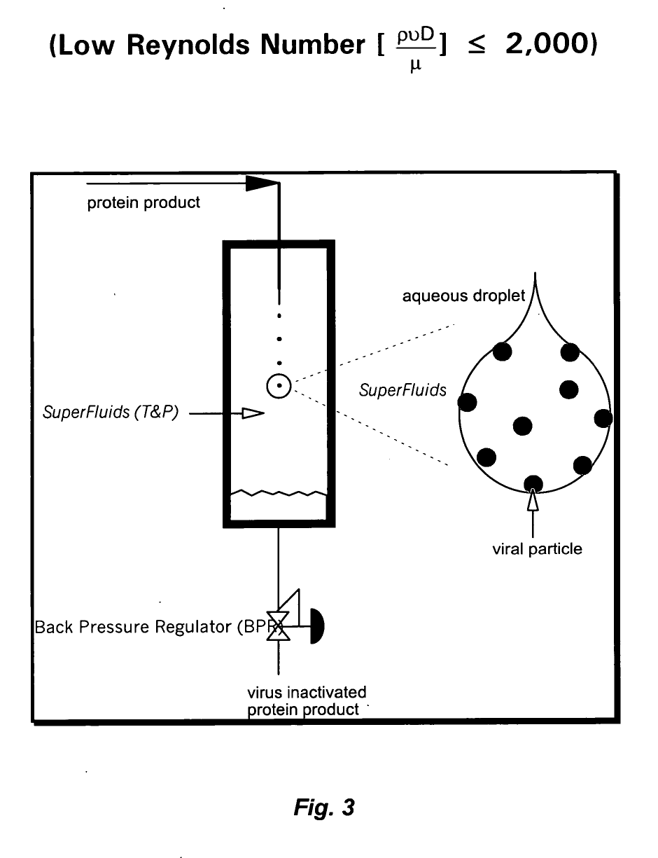

FIG. 4 is a multistage laminar flow SCoNCF CFI system.

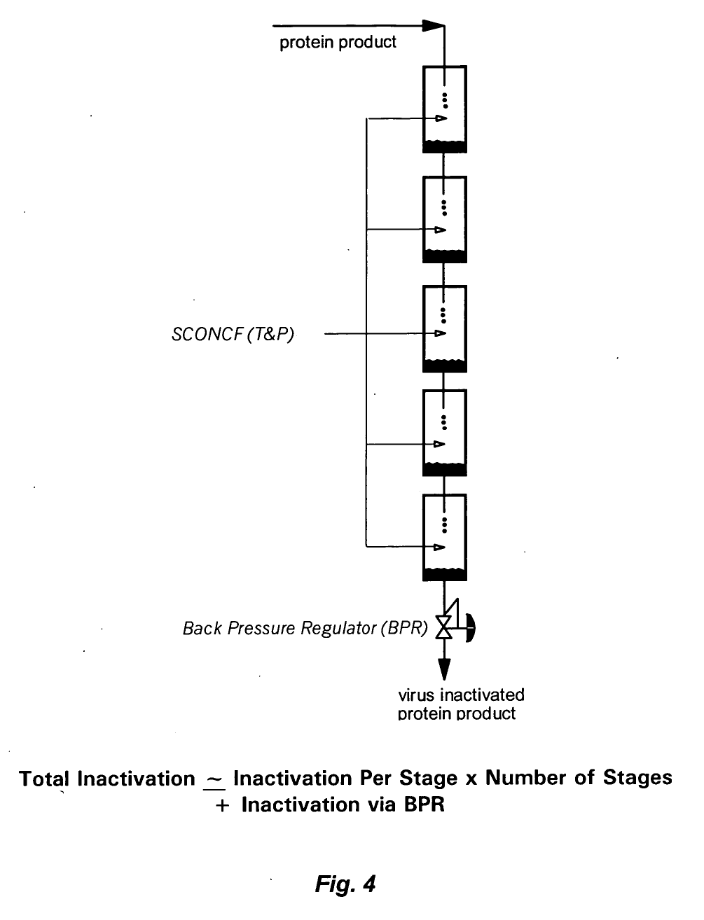

FIG. 5 is a single-stage laminar flow SCoNCF CFI apparatus.

FIG. 6 is a two-stage laminar flow SCoNCF CFI apparatus.

FIG. 7 is a bar graph of log reduction of EMC versus temperature at 5,000 psig.

FIG. 8 is a bar graph of log reduction of EMC versus pressure at 50° C.

FIG. 9 is a bar graph of the inactivation of HIV-1 by different SCoNCF at 3,000 psig and 22° C. Virus-containing supernatant was diluted 1:10 in RPMI and run through the CFI-unit with different SCoNCF conditions. HIV-1Δtat-rev was used for each run. For each experiment, an aliquot was not exposed to SCoNCF and served as a time and temperature (t&T) control. 10-fold serial dilutions of the control and treated samples were made and used in the TCID₅₀ assay to measure infectious virus. The Log Inactivation was calculated by subtracting the log TCID₅₀/ml of the t&T from the log TCID₅₀/ml of the CFI-Treated sample. N₂O/CO₂—N₂O with trace quantities of CO₂, 23 ppm; N₂O+5% CO₂—a mixture of 95% N₂O and 5% CO₂ by volume; White arrows indicate that the Log Inactivation is greater than the indicated value (log TCID₅₀/ml of the CFI-Treated sample was at the limit of detection).

FIG. 10 is a bar graph showing that SCoNCF-treated FCS is an effective serum for cell growth. HeLa (red squares), A549 (blue triangles), and 3T6 (green circles) were incubated with either untreated (closed symbols) or SCoNCF-treated (open symbols) FCS and monitored for growth by counting cells with a hemocytometer. N₂O/CO₂ at 2,000 psig and 22° C. was used to generate SCoNCF-treated FCS.

### DETAILED DESCRIPTION OF PREFERRED EMBODIMENTS

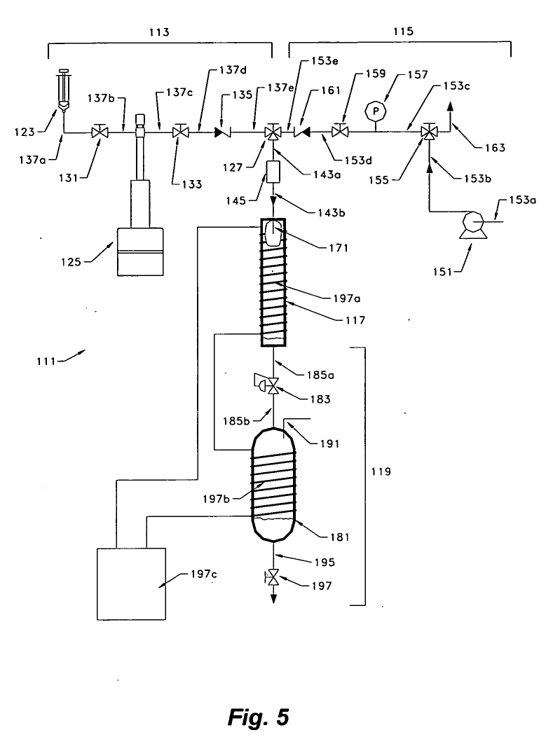

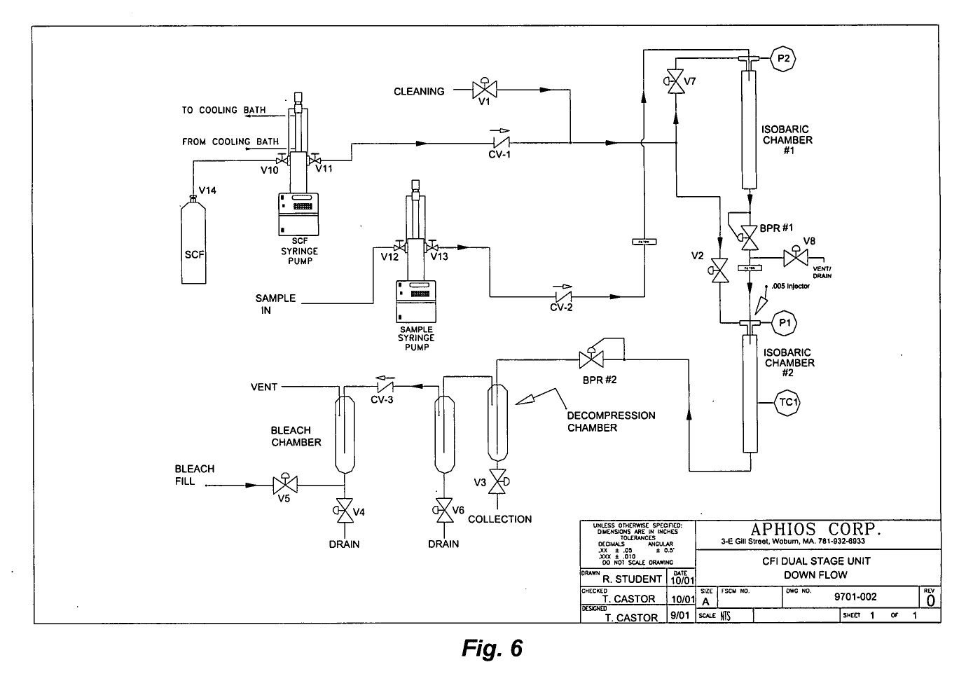

A single-stage laminar flow diffusion mixing CFI test apparatus is shown in FIG. 5. A two-stage laminar flow diffusion mixing CFI test apparatus is shown in FIG. 6. Injection of sample is performed by a 0.005″ internal diameter (ID) tube. Steady sample flow into the system is provided by the Isco syringe pump. Flow out of the system is regulated by a Tescom valve. Sample flow rates up to about 10 ml/min are possible without transition to turbulent flow in the 0.005″ ID tube.

Operation begins by charging the system with SCoNCF. This is done by the SCF syringe pump through valves V-11, V-7 and V-2. When the system pressure is close to the desired value, the sample syringe pump is run in the constant flow-rate mode at 4.0 ml/min, supplying sample to the isobaric chambers. After a few milliliters are supplied to the isobaric chambers, the backpressure regulators, BPR-1 and BPR-2, are adjusted to operating pressure. The sample is degassed in a collection chamber and withdrawn from V-6.

We have also discovered that the SCoNCF type is also important. After testing several different SCoNCF for their efficacy of inactivating virus while preserving integrity, we have discovered that nitrous oxide (N₂O) with trace quantities of carbon dioxide (CO₂) is quite efficacious in inactivating viruses while preserving protein integrity. N₂O/CO₂ is nitrous oxide with 10 to 10,000 ppm carbon dioxide. We discovered that cell growth was maintained after treatment with SCoNCF N₂O/CO₂, suggesting that this mixture did not adversely impact the cells, proteins, enzymes and growth factors responsible for cell growth. We also discovered that SCoNCF N₂O/CO₂ was very effective in inactivating the enveloped virus HIV and the small, tough, nonenveloped virus, parvovirus B19.

### EXAMPLES

Several examples are included to provide representative data on SCoNCF critical fluid inactivation (CFI) of both enveloped and non-enveloped viruses in various proteinaceous materials, with maintaining biological activity. In a typical experiment, the selected proteinaceous matrix (including fetal bovine serum, plasma or plasma products, such as immunoglobulins) is spiked with a particular virus and treated using the bench scale SCoNCF CFI equipment shown in FIGS. 5 and 6 or appropriate modifications under tightly controlled conditions with defined SCoNCF, temperature and pressure. The residence time of droplet in a single stage laminar flow CFI unit is approximately 20 seconds; the residence time in a two-stage unit is approximately 40 seconds. Treated samples are collected either in bulk at the end of a complete run or at specified times during the run. Control and treated materials are analyzed for residual virus. Samples are also evaluated with respect to total protein and biological properties of the proteins.

### Example 1

Several tests were performed with murine-C retrovirus (MuLV), and nitrous oxide at 2,200 psig and 22° C. MuLV, an enveloped or lipid-encased virus that has an outer diameter of approximately 100 nanometers (nm), is often used as a surrogate for the human immunodeficiency virus (HIV). Selected results are presented in Table 1.

TABLE 1
SCONCF CFI INACTIVATION OF MURINE LEUKEMIA
VIRUS (MULV) WITH NITROUS OXIDE IN
LAMINAR FLOW INJECTION UNIT

| Parameters | CFI-286 | CFI-380 | CFI-381 | CFI-464 |
|---|---|---|---|---|
| Pressure (psig) | 2,000 | 2,000 | 2,000 | 2,000 |
| Temperature (° C.) | 22 | 22 | 22 | 22 |
| Time (mins) | <1 | <1 | <1 | <1 |
| Titer Control | 1 × 10^{4.0} | 1 × 10^{6.0} | 1 × 10^{3.0} | 1 × 10^{5.5} |
| Titer After | 1 × 10^{3.0} | 1 × 10^{3.7} | 1 × 10^{1.0} | 0 × 10^{0.0*} |
| −log₁₀ reduction | 1.0 | 2.3 | 2.0 | >5.5 |
| No. of Stages | 0 | 1 | 1 | 2 |

*below minimum detection level

CFI-286 was performed by directly passing the pressurized stream through the backpressure regulator without having contacted that stream with nitrous oxide. This zero (0) stage experiment resulted in about 1 log inactivation. Experiments CFI-380 and CFI-381 were performed in a single stage laminar flow CFI unit in the presence of nitrous oxide under similar conditions of temperature and pressure for less than one minute. These experiments resulted in about 2 logs of MuLV inactivation in about 20 seconds. Experiment CFI-464 was conducted in a two-stage laminar flow CFI unit with nitrous oxide under identical conditions of temperature and pressure. This two-stage experiment resulted in greater than 5.5 logs of MuLV inactivation. The two-stage unit inactivated about twice the amount of MuLV inactivated by the one stage unit plus one log due to the decompression valve in a residence time of less than one minute. This discovery shows that the laminar flow CFI unit is effective in very short times (<20 seconds) and is directly scalable on a per stage basis so that the levels of inactivation can be controlled by the number of stages in place.

### Example 2

Several tests were performed with vesicular stomatitis virus (VSV) and nitrous oxide at 2,200 psig and 22° C. VSV is an enveloped virus with a distinctive bullet shape (50-95 nm×130-380 nm). VSV is a member of the Rhabdovirus family. VSV possess a negative-strand RNA genome and codes for only five proteins that are found in the virion. VSV is an animal pathogen that grows well in cell culture; the host cell for VSV is the A549 cell line. Quantitation was carried out using an infectivity titration assay (50% end point referred to as TCID50); titration was performed on overnight cultures of A549 host cells. Selected results are presented in Table 2.

TABLE 2
SCONCF CFI INACTIVATION OF VESICULAR
STOMATITIS VIRUS (VSV) WITH NITROUS OXIDE
IN LAMINAR FLOW INJECTION UNIT

| Parameters | CFI-574 | CFI-588 |
|---|---|---|
| Pressure (psig) | 4,000 | 4,000 |
| Temperature (° C.) | 40 | 40 |
| Time (mins) | <1 | <1 |
| Titer Control | 1 × 10^{5.0} | 1 × 10^{5.5} |
| Titer After | 1 × 10^{2.5} | 0 × 10^{0.0*} |
| −log₁₀ reduction | 2.5 | >5.5 |
| No. of Stages | 1 | 2 |

*below minimum detection level

In a two-stage unit, the SCoNCF CFI process achieved about twice the inactivation shown in the single stage unit. Other data for the inactivation of VSV by nitrous oxide in shows that inactivation increased with increases in temperature and pressure. An average of 4 logs of inactivation were achieved with nitrous oxide at a pressure of 5,000 psig and a temperature of 40° C. At the same pressure but a lower temperature of 22° C., about one half or 2 logs of inactivation are achieved suggesting, that the rate of inactivation is very sensitive to temperature. At lower temperatures (15° C. and 22° C.), inactivation of VSV does not appear to be very sensitive to pressure.

### Example 3

Several experiments were conducted with encephalomyocarditis (EMC), a tough, prototypical non-enveloped or protein-encased virus with different SCoNCF at different pressures and temperatures in the single stage laminar flow unit. EMC, a member of the Picomaviridae family, is a positive-strand RNA virus, which lacks an envelope. EMC is icosahedral in shape with a size of 20 to 30 nanometers. EMC, an animal virus that is non-pathogenic to man, is often used as a surrogate for Hepatitis A and a marker virus in process validation studies. Other viruses of major concern belonging to the Picomaviridae family include Hepatitis A, Polioviruses and Parvoviruses. Quantitation was carried out using an infectivity titration assay (50% end point referred to as TCID50) on susceptible host cells A549, a cell line derived from human carcinoma tissue. A sample of the experimental results is listed in Table 3.

TABLE 3
SCONCF CFI INACTIVATION OF ENCEPHALOMYOCARDITIS
(EMC) WITH FREON-22 IN SINGLE-STAGE
LAMINAR FLOW INJECTION UNIT

| Parameters | CFI-887 | CFI-551 | CFI-914 | CFI-915 |
|---|---|---|---|---|
| Pressure (psig) | 3,000 | 3,000 | 3,000 | 3,000 |
| Temperature | 50 | 50 | 50 | 50 |
| (° C.) | | | | |
| Time (mins) | <1 | <1 | <1 | <1 |
| Titer Control | 1 × 10^{5.6} | 1 × 10^{5.6} | 1 × 10^{5.2} | 1 × 10^{5.2} |
| Titer After | 1 × 10^{-0.3} | 1 × 10^{0.2} | 1 × 10^{-0.5} | 1 × 10^{-0.4} |
| −log₁₀ reduction | 5.9 | 5.4 | >5.7* | 5.6 |
| No. of Stages | 1 | 1 | 1 | 1 |

*below minimum detection level

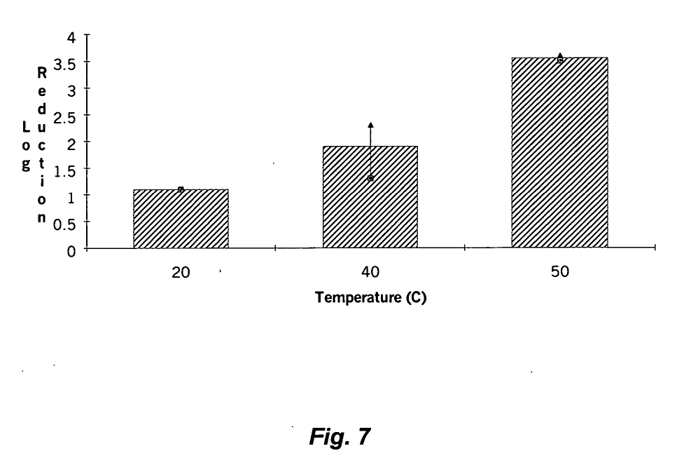

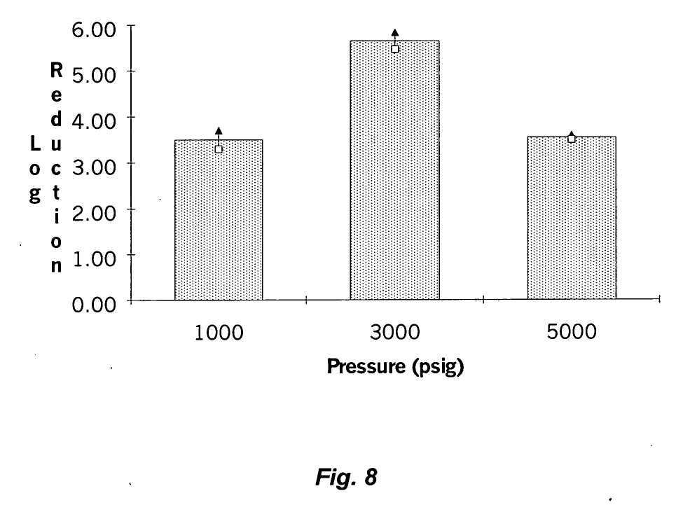

As shown in Table 3, approximately six logs of the tough, prototypical non-enveloped EMC virus were inactivated by Freon-22 in a single stage laminar flow injector SCoNCF CFI prototype apparatus in less than 20 seconds. Other experiments in the single-stage, laminar flow injector CFI unit indicate the following: (1) EMC inactivation (on the average 5.7 logs) was optimal with Freon-22 at 3,000 psig and 50° C. in a single stage laminar flow unit. This was consistently confirmed in at least four experiments, CFI-887, CFI-889, CFI-914 and CFI-915; (2) As shown in FIG. 7, inactivation increases with temperature increase—˜1 log for every 10 ° C. increase in temperature with Freon-22 at 5,000 psig; and (3) As shown in FIG. 8, inactivation is greatest at a pressure of 3,000 psig with Freon-22 at 50° C. This result was totally unanticipated since it was expected that further increases in pressure would result in higher explosive decompression forces or more effectively disrupt virions resulting in greater virus kill.

### Example 4

The inactivation of several viruses in Freon-22 at 3,000 psig and 50 ° C., conditions, which appear to be optimum for inactivating EMC, are listed in Table 4. All experiments were conducted with an Isco syringe pump with the exception of CFI-908 and CFI-909, for Hepatitis A (HAV), which were conducted with the Eldex piston pump at 4 ml/min. The latter course of action was taken because the Eldex pump can be operated in the laminar flow safety cabinet, which would contain any aerosols generated. The data listed in Table 4 indicates the following trends:
All of the non-enveloped virus, Human Adenovirus, Type 5 was consistently inactivated (>5.1 and 5.3 logs) with Freon-22 at 3,000 psig and 50° C.
In excess of four logs of inactivation (4.1 and 4.2) were achieved with the very small and tough Poliovirus in less than 20 seconds with Freon-22 at 3,000 psig and 50° C.
Approximately one log of inactivation was obtained for Hepatitis A (HAV) virus with Freon-22 at 3,000 psig and 50° C. Further testing as a function of pressure, temperature and SCoNCF type will be required to improve the inactivation of HAV per laminar flow stage. The ˜1 log of inactivation of HAV for the single stage unit is, however, sufficient to meet our design criteria since 5 stages are planned for commercial-scale units.
Consistent one log kill (0.9 and 1.0 logs) was achieved with the tough, non-enveloped Reovirus with Freon-22 at 3,000 psig and 50° C.
Of all the enveloped viruses tested with Freon-22 at 3,000 psig and 50° C., Bovine Diarrhea Virus (BVD) was the least effected with 2.3 logs kill in a single-stage laminar flow injection unit. Unlike the other enveloped viruses, which had complete or near-complete inactivation, BVD was only partially inactivated. However, these results were significant for a single-stage continuous laminar flow CFI unit.
Complete inactivation of greater than six logs (>6.5 and >6.6) was obtained with Vesicular Stomatitis Virus (VSV) in Freon-22 at 3,000 psig and 50° C. This was the greatest single-stage inactivation of VSV in a continuous laminar flow CFI unit.
Complete or near-complete inactivation of greater than six logs (>6.5 and 6.5) was also obtained with Sindbis in Freon-22 at 3,000 psig and 50° C. This was the greatest inactivation of Sindbis under any conditions or in any CFI unit.

Complete inactivation of greater than 2 logs (>2.5 and >2.6) was achieved with TGE in Freon-22 at 3,000 psig and 50° C. The viral titer of the TGE used was low so that TGE inactivation could have been better than suggested by the results.

TABLE 4
SCONCF CFI INACTIVATION OF DIFFERENT VIRUSES BY FREON-22 @
3,000 PSIG AND 50° C. IN SINGLE-STAGE LAMINAR FLOW
INJECTION UNIT

| CFI | | | Virus Type | | | | |
|---|---|---|---|---|---|---|---|
| No. | Virus | Matrix | Family | Genome | Size | Capsid | −log₁₀ Kill |
| 916 | Adeno | FBS | Adenoviridae | ds-DNA | 70-90 | Non-Env. | >5.3 |
| 917 | Adeno | FBS | Adenoviridae | ds-DNA | 70-90 | Non-Env. | >5.1 |
| 918 | Polio | FBS | Picornaviridae | ss-RNA | 18-26 | Non-Env. | 4.1 |
| 919 | Polio | FBS | Picornaviridae | ss-RNA | 18-26 | Non-Env. | 4.2 |
| 908 | HAV | FFP | Picornaviridae | ss-RNA | 24-30 | Non-Env. | 1.3 |
| 909 | HAV | FFP | Picornaviridae | ss-RNA | 24-30 | Non-Env. | 1.0 |
| 898 | Reo | FBS | Reoviridae | ds-RNA | 65-75 | Non-Env. | 0.9 |
| 889 | Reo | FBS | Reoviridae | ds-RNA | 65-75 | Non-Env. | 1.0 |
| 904 | VSV | FBS | Rhabdoviridae | ss-RNA | 60-180 | Enveloped | >6.5 |
| 905 | VSV | FBS | Rhabdoviridae | ss-RNA | 60-180 | Enveloped | >6.6 |
| 906 | Sindbis | FBS | Togaviridae | ss-RNA | 60-70 | Enveloped | >6.5 |
| 907 | Sindbis | FBS | Togaviridae | ss-RNA | 60-70 | Enveloped | 6.5 |
| 902 | TGE | FBS | Coronaviridae | ss-RNA | 80-130 | Enveloped | >2.5 |
| 903 | TGE | FBS | Coronaviridae | ss-RNA | 80-130 | Enveloped | >2.6 |
| 900 | BVD | HS | Togaviridae | ss-RNA | 60-70 | Enveloped | 2.3 |
| 901 | BVD | HS | Togaviridae | ss-RNA | 60-70 | Enveloped | 2.3 |

### Example 5

From the data listed and discussed in the examples above, Freon-22 (hydrodifluorochloromethane—CHCLF2) appears to have very virucidal properties for both major classes of viruses, enveloped and non-enveloped. Relative to other chlorofluorcarbons such as Freon-11 and Freon-12 which are being banned by the 1988 Montreal protocol, Freon-22 is very stable and only has a slight ozone depletion potential (0.05 ODP) because it has a hydrogen atom in its structure. Even though Freon-22 has an ODP that is twenty times less than Freon-11, Freon-22 cannot be used in any new applications after 2010 and in any existing applications after 2020 in accordance with the 1988 Montreal protocol.

Since Freon-22 use and production may be adversely impacted by future environmental concerns, we are accelerating the search for alternate refrigerants. In the first step of this process, we evaluated the impact of alternate refrigerants on the prototypical, non-enveloped EMC virus at conditions found optimal for Freon-22. The second step would be to find optimal conditions for the best available alternate. The third step will be to evaluate the impact of these conditions on a range of non-enveloped and enveloped viruses. The thermodynamic properties of Freon-22 and the tested alternate refrigerants are listed in Table 5. The results of the comparative first steps are listed in Table 6.

TABLE 5
THERMODYNAMIC PROPERTIES OF
SELECTED FLUOROCARBONS

| | | Critical | Critical | |
|---|---|---|---|---|
| | Chemical | Temperature | Pressure | Dipole |
| Generic Name | Formula | T_{c} (C.) | P_{c} (psig) | Moment |
| Freon-22 | CHClF2 | 96.0 | 707.2 | 1.4 |
| Freon-23 | CHF3 | 25.9 | 686.5 | 1.6 |
| HCFC-123 | CF3CHCl2 | 183.6 | 532.0 | 1.36 |
| HCFC-124 | CHClFCF3 | 122.2 | 524.5 | 1.47 |
| HCFC-134a | CH2FCF3 | 101.1 | 574.2 | 2.06 |

From the comparison in Table 6, Freon-23 (trifluoromethane—CHF3) appears to be the best alternate to Freon-22. On the average, Freon-23 inactivated ˜3 logs (2.2 and 3.5) versus ˜6 logs (5.9, 5.4, >5.7 and 5.6) of EMC at similar conditions of temperature (50° C.) and pressure (3,000 psig). Per the listing of thermodynamic properties in Table 5, Freon-23 appears to be an excellent CFI candidate because: (i) it is non-chlorinated (the chlorine component of chlorofluorocarbons is thought to be responsible for their negative impact on the ozone layer): (ii) has a low critical temperature of 25.9° C. (allows operation close to critical conditions while minimizing thermal denaturation of biological proteins); and (iii) has a relatively large dipole moment of 1.6 debyes (has a large potential of solubilizing polar lipids and fats).

TABLE 6
SCONCF CFI INACTIVATION OF ENCEPHALOMYOCARDITIS (EMC) VIRUS
IN SINGLE-STAGE LAMINAR FLOW INJECTION UNIT WITH DIFFERENT
FLUOROCARBONS

| | | | Critical | | | Press | Temp | |
|---|---|---|---|---|---|---|---|---|
| CFI No. | Virus | Matrix | Fluid | Mixing | Time (mins) | (psig) | (° C.) | −log₁₀ Kill |
| 887 | EMC | FBS | Fr-22 | Laminar | 0.33 | 3,000 | 50 | 5.9 |
| 889 | EMC | FBS | Fr-22 | Laminar | 0.33 | 3,000 | 50 | 5.4 |
| 914 | EMC | FBS | Fr-22 | Laminar | 0.33 | 3,000 | 50 | >5.7 |
| 915 | EMC | FBS | Fr-22 | Laminar | 0.33 | 3,000 | 50 | 5.6 |
| 926 | EMC | FBS | HFC-134a | Laminar | 0.33 | 3,000 | 50 | 1.3 |
| 927 | EMC | FBS | HFC-134a | Laminar | 0.33 | 3,000 | 50 | 0.1 |
| 933 | EMC | FBS | HFC-134a | Laminar | 0.33 | 3,000 | 50 | 0.6 |
| 932 | EMC | FBS | HFC-134a | Laminar | 0.33 | 5,000 | 50 | 0.3 |
| 928 | EMC | FBS | Fr-124 | Laminar | 0.33 | 3,000 | 50 | 0.5 |
| 929 | EMC | FBS | Fr-124 | Laminar | 0.33 | 3,000 | 50 | 0.4 |
| 930 | EMC | FBS | Fr-23 | Laminar | 0.33 | 3,000 | 50 | 2.2 |
| 931 | EMC | FBS | Fr-23 | Laminar | 0.33 | 3,000 | 50 | 3.5 |

### Example 6

A set of experiments conducted to find optimal conditions for Freon-23 are listed in Table 7.

TABLE 7
SCONCF CFI INACTIVATION OF ENCEPHALOMYOCARDITIS
(EMC) VIRUS IN SINGLE-STAGE LAMINAR FLOW INJECTION
UNIT WITH FREON-23 AT DIFFERENT CONDITIONS OF T & P

| | | | Crit- | | | | | |
|---|---|---|---|---|---|---|---|---|
| CFI | | | ical | | Time | Press | Temp | −log₁₀ |
| No. | Virus | Matrix | Fluid | Mixing | (mins) | (psig) | (° C.) | Kill |
| 936 | EMC | FBS | Fr-23 | Laminar | 0.33 | 1,000 | 50 | 2.7 |
| 937 | EMC | FBS | Fr-23 | Laminar | 0.33 | 1,000 | 50 | 3.5 |
| 930 | EMC | FBS | Fr-23 | Laminar | 0.33 | 3,000 | 50 | 2.2 |
| 931 | EMC | FBS | Fr-23 | Laminar | 0.33 | 3,000 | 50 | 3.5 |
| 934 | EMC | FBS | Fr-23 | Laminar | 0.33 | 5,000 | 50 | 2.7 |
| 935 | EMC | FBS | Fr-23 | Laminar | 0.33 | 5,000 | 50 | 3.1 |
| 938 | EMC | FBS | Fr-23 | Laminar | 0.33 | 3,000 | 26 | 0.2 |
| 943 | EMC | FBS | Fr-23 | Laminar | 0.33 | 3,000 | 37 | 0.0 |
| 941 | EMC | FBS | Fr-23 | Laminar | 0.33 | 5,000 | 58 | 4.6 |
| 931 | EMC | FBS | Fr-23 | Laminar | 0.33 | 5,000 | 58 | 4.5 |

Interestingly, the data for CFI-936, 937, 930, 931, 934 and 935 suggest that the inactivation of the tough, non-enveloped EMC virus by Freon-23 is independent of pressure over the narrow range of pressures tested (1,000 to 5,000 psig) at 50° C. This finding is very significant since operating a low pressure would significantly reduce the initial capital as well as operating costs of SCoNCF CFI viral inactivation equipment. This data differs from that of Freon-22, which indicate the inactivation of EMC by Freon-22 appears to have a maxima at 3,000 psig over the same range of pressure.

The data in Table 7 indicates that the inactivation of EMC by Freon-23 is very sensitive to temperature, with little or no inactivation at lower temperatures (26° C. and 37° C.) and improved inactivation at 58° C. The data sets for both Freon-22 and Freon-23 indicate that activation of EMC increases with temperature.

### Example 7

In Table 8, single-stage and two-stage CFI experiments on EMC with Freon-22 are listed. The experiments, performed at 5,000 psig and 50° C., were based on initial EMC viral inactivation results at these conditions in the single-stage CFI unit (CFI-882 and CFI-883).

TABLE 8
SCONCF CFI INACTIVATION OF ENCEPHALOMYOCARDITIS
(EMC) WITH FREON-22 IN SINGLE-STAGE AND
TWO-STAGE LAMINAR FLOW INJECTION UNITS

| Parameters | CFI-882 | CFI-883 | CFI-894 | CFI-895 |
|---|---|---|---|---|
| Pressure (psig) | 5,000 | 5,000 | 5,000 | 5,000 |
| Temperature (° C.) | 50 | 50 | 50 | 50 |
| Time (mins) | <1 | <1 | <1 | <1 |
| Titer Control | 1 × 10^{5.7} | 1 × 10^{5.5} | 1 × 10^{5.5} | 1 × 10^{5.8} |
| Titer After | 1 × 10^{2.1} | 1 × 10^{2.0} | 1 × 10^{0.6} | 1 × 10^{1.6} |
| −log₁₀ reduction | 3.6 | 3.5 | 4.9 | 4.2 |
| No. of Stages | 1 | 1 | 2 | 2 |

The data listed in Table 8 indicate that over four logs of inactivation (4.9 and 4.2 logs) was obtained with EMC in the two-stage CFI unit. In the single-stage unit (CFI-882 and CFI-883) 3.6 and 3.5 logs were obtained. So, the second stage appears to add an average of one log of inactivation.

### Example 8

Several aliquots of a hyper-immunoglobulin were treated in a single stage laminar flow injection unit under various conditions of temperature (20° C. to 40° C.) and pressure (3,000 to 4,000 psig) with SCoNCF nitrous oxide. Biochemical and biological analysis of the CFI treated samples were carried out and compared to a non-processed sample for molecular integrity and biological activity. The results of some of the analyses are tabulated in Table 9.

TABLE 9
SCONCF CFI TREATMENT OF HYPER-IMMUNOGLOBULIN
IN SINGLE-STAGE LAMINAR FLOW INJECTION UNIT

| | HPLC-SEC | | Protein | ELISA |
|---|---|---|---|---|
| CFI No. | (%) | Anti-Complementary | (mg/ml) | MEP Abs |
| 595A | 104.3 | >1.81 | 18.00 | 351.4 |
| 595B | 99.7 | >1.78 | 17.84 | 385.4 |
| 596 | 108.1 | >1.78 | 17.78 | 346.2 |
| 597A | 101.4 | >1.83 | 18.27 | 349.7 |
| 597B | 92.7 | >1.77 | 17.65 | 313.8 |
| 598 | 93.7 | >1.76 | 17.58 | 325.8 |
| 599A | 94.7 | >1.74 | 18.14 | 379.5 |
| 599B | 95.2 | >1.74 | 17.39 | 370.8 |
| 600 | 93.1 | >1.82 | 18.20 | 374.2 |

Protein and anti-MEP antibodies content were determined by Bradford assay and ELISA assay, respectively, and were consistent with experimental control data. Molecular integrity of the treated samples was determined by reducing and non-reducing SDS-PAGE, HPLC-SEC, and Anti-complementary activity. The SDS-PAGE analysis of the experimental control and the treated process samples display similar banding patterns. The processed samples exhibited no significant aggregate or fragment bands, as compared to the experimental control. Repeated HPLC-SEC analyses showed that the treated samples exhibited similar chromatographic profiles to the untreated at 280 nm, and that there did not appear to be any significant aggregation or fragmentation. The process samples showed no significant aggregate formation that could be detected by the anti-complimentary activity, relative to the experimental control. Biological activities of the treated samples were measured by the Opsonophagocytosis Potency assay. All treated samples appear to exhibit higher specific opsonic activities than the experimental control.

### Example 9

Several aliquots of an intravenous immunoglobulin were treated in a single stage laminar flow injection unit under various conditions of temperature (22° C. to 50° C.) and pressure (2,000 to 5,000 psig) with SCoNCF Freon-22. Biochemical and biological analysis of the CFI treated samples were carried out and compared to a non-processed sample for molecular integrity and biological activity. The results of some of the analyses are tabulated in Table 10.

TABLE 10
SCONCF CFI TREATMENT OF IMMUNOGLOBULIN (IV)
IN SINGLE-STAGE LAMINAR FLOW INJECTION UNIT

| CFI No. | RSV | POLIO | MEASLES | TETANUS | DIPHTHERIA |
|---|---|---|---|---|---|
| Control | 2186 | 2.4 | 1.3 | 311 | 4.8 |
| 752 | 2262 | 2.4 | 1.3 | 306 | 4.8 |
| 753 | 1870 | 2.4 | 1.3 | 285 | 4.8 |
| 754 | 2491 | 1.6 | 1.8 | 286 | 4.8 |
| 755 | 2142 | 1.6 | 1.5 | 290 | 4.8 |
| 756 | 982 | 0.8 | 1.4 | 295 | 4.8 |
| 757 | 1424 | 1.5 | 1.1 | 303 | 4.8 |

Antibody assays to asses IgG antigen binding and antibody effect or functions include: (1) neutralization of RSV, polio and measles viruses; (2) neutralization of tetanus and diphtheria bacterial toxins; and (3) ELISA measurement of antigen binding. In most cases, there was no significant difference between the CFI treated samples and the control. HPLC, Nephalometry and Anti-Complimentary activity assays all indicated that the treated samples had retained their molecular integrity.

### Example 10

Preliminary CFI experiments were conducted on fresh porcine plasma in order to evaluate the impact of CFI conditions on coagulation factors. Fresh, citrated porcine whole blood was shipped on wet ice by an overnight express delivery service from Pel-Freez Biologicals, Rogers, Ark. The whole blood was centrifuged to separate the red blood cells from the plasma that was snap-frozen and stored at −80° C. The fresh, frozen porcine plasma was thawed at 30C and treated in the BTCF unit with nitrous oxide at 21° C. and 1,200 psig at different sample flowrates of 8(A), 8(B), 2(C), and 6(D) ml/minute. Control, untreated, and treated samples were stored at −80° C. prior to analysis. When ready to be analyzed, samples were thawed and analyzed for total protein, pH, enzymes, coagulation proteins, prothrombin and activated prothrombin times—all of which were tested in duplicate. The data, listed in Table 11, indicate little or no change in pH, fibrinogen, Factor VIII or Factor XI after CFI treatment. Prothrombin and activated prothrombin times of CFI treated samples were within ±3.0 seconds of the control time.

TABLE 11
SCONCF CFI TREATMENT OF FRESH FROZEN
PORCINE PLASMA IN SINGLE-STAGE
LAMINAR FLOW INJECTION UNIT

| | pH | % Fibrinogen | % Factor VIII | % Factor XI |
|---|---|---|---|---|
| Control | 7.75 | 100 | 100 | 100 |
| Treated Sample A | 7.40 | 120 | 106 | 115 |
| Treated Sample B | 7.83 | 113 | 140 | 101 |
| Treated Sample C | 8.03 | 80 | 106 | 108 |
| Treated Sample D | 7.95 | 113 | 122 | 118 |

### Example 11

Several experimental runs were performed on fresh frozen (human) plasma (FFP)in the single-stage laminar flow SCoNCF CFI unit with nitrous oxide (N₂O). Temperature and pressure were varied for each experimental run. All SCoNCF CFI treated samples, as well as untreated time and temperature controls, mechanical controls (sample pumped through the unit at a specified temperature and at no pressure and without any SCoNCF), and pretreated controls were analyzed for protein integrity. Protein integrity. was measured by the Pierce BCA protein assay, Activated Prothrombin Time (APTT), pH, and Factor VIII. A sample of these results are presented in Table 12.

TABLE 12
IMPACT OF SCONCF CFI ON FRESH FROZEN HUMAN PLASMA
IN SINGLE-STAGE LAMINAR FLOW INJECTION UNIT

| Parameters | CFI-676 | CFI-679 |
|---|---|---|
| Pressure (psig) | 2,000 | 5,000 |
| Temperature (° C.) | 37 | 15 |
| Time (mins) | <1 | <1 |
| % Factor VIII | 87 | 84 |
| % Total Protein | 94 | 100 |

As shown in Table 12, excessive FVIII protein damage during the SCoNCF CFI process was not observed and labile protein recovery was well above 80% of untreated time and temperature controls. Hydrogen ion concentration and total proteins of SCoNCF CFI treated FFP do not appear to be significantly adversely affected. Other testing indicated that the SCoNCF CFI process had little or no effect on sensitive blood plasma proteins. Recovery of protein activity in comparison to the time and temperature controls ranged between 76% and 92% for Factor VIII, 85% and 92% for α₁-PI, and 91% and 95% for ATIII. Recovery of protein was worst at 15° C./2,500 psig, and somewhat better at 37° C./5,000 psig. In conclusion, treatment of source human plasma with SCoNCF appears to produce minimal damage to plasma proteins.

### Example 12

To determine the effect of different SCoNCF on HIV inactivation, HIVΔtat-rev-supernatant, from infected CEM-TART cells, was thawed the day of the experiment and diluted 1:10 in RPMI. Diluted virus was used immediately or kept at 4° C. A sample of diluted virus was held at the same temperature for the same time (t&T control) as that applied to the CFI unit. After the run, the tissue culture infectious dose 50 (TCID₅₀) assays for the t&T control and CFI-treated samples were conducted to measure infectious virus. It was noted that cells at the top dilution of virus (1:10) did not grow for some SCoNCF conditions, and therefore were not included when calculating the TCID₅₀. The Log Inactivation was calculated by subtracting the log₁₀ TCID₅₀/ml of the CFI-treated sample from the log₁₀ TCID₅₀/ml of the t&T control.

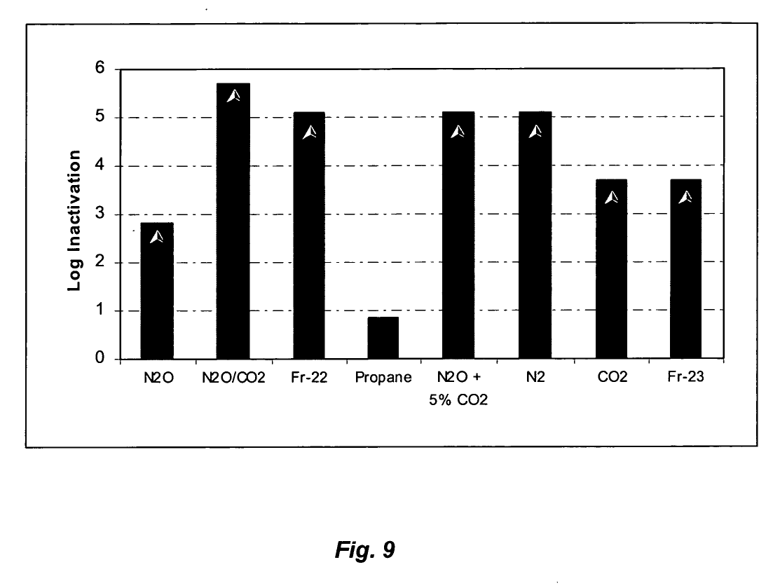

The results of eight experiments using different SCoNCF: N₂O, N₂O/CO₂ (N₂O with trace quantities of CO₂, 23 parts per million (ppm)), Freon-22, Propane, N₂O +CO₂ (a mixture of 95% N₂O and 5% CO₂ by volume), N₂, CO₂ and Freon-23 in a single-stage laminar flow unit are summarized in Table 13 and shown in FIG. 9.

TABLE 13
INACTIVATION OF HIV-1 BY DIFFERENT SCONCF AT 3,000 PSIG
AND 22° C. IN A SINGLE-STAGE LAMINAR FLOW
SCONCF CFI UNIT

| | | | | Log₁₀ | |
|---|---|---|---|---|---|
| | | | Log₁₀ | TCID₅₀/ | |
| | | | TCID₅₀/ | ml | −Log₁₀ |
| Exp. | | | ml | (CFI- | In- |
| No. | SCONCF | Virus | (t&T)^{c} | treated | activation^{d} |
| VAC-5 | N₂O | HIV-1Δtat-rev^{b} | 2.8 | undetected | >2.8 |
| VAC-6 | N₂O/CO₂^{a} | HIV-1Δtat-rev^{b} | 5.7 | undetected | >5.7 |
| VAC-8 | Fr-22 | HIV-1Δtat-rev^{b} | 5.1 | undetected | >5.1 |
| VAC-9 | C₃H₈ | HIV-1Δtat-rev^{b} | 5.0 | 4.1 | 0.9 |
| VAC-10 | N₂O/5% | HIV-1Δtat-rev^{b} | 5.1 | undetected | >5.1 |
| | CO₂ | | | | |
| VAC-11 | N₂ | HIV-1Δtat-rev^{b} | 5.1 | undetected | >5.1 |
| VAC-12 | CO₂ | HIV-1Δtat-rev^{b} | 3.7 | undetected | >3.7 |
| VAC-13 | Fr-23 | HIV-1Δtat-rev^{b} | 3.7 | undetected | >3.7 |

^{a}N₂O/CO₂ —N₂O with trace quantities of CO₂
^{b}Virus-containing supernatant was diluted 1:10 in RPMI (total of 20 ml feed volume) and run through the CFI-unit with different SCoNCF
^{c}Time and temperature control
^{d}−(log₁₀ TCID₅₀/ml of CFI-treated −log₁₀ TCID₅₀/ml of untreated control)

### Example 13

To determine the presence of a major capsid protein of HIV after treatment with SCoNCF the amount of p24 in the t&T control and the CFI-treated samples for each SCoNCF was determined by ELISA (Table 14). Slightly higher amounts of p24 were detected in the CFI-samples treated with N₂O, C₃H₈, N₂, CO₂, and Fr-23 as compared to the t&T control samples. This may indicate leaking of p24 out of a compromised virion or enhanced exposure of the core proteins and nucleic acids. In other cases such as samples treated with N₂O/CO₂ (N₂O with 23 ppm CO₂), Fr-22, and N₂O/5% CO₂ in which changes p24 was negligible or nonexistent, CFI treatment may have resulted in relatively intact virion.

TABLE 14
EFFECT OF DIFFERENT SCONCF AT 3,000 PSIG AND 22° C.
ON HIV-1 P24 IN THE SINGLE-STAGE LAMINAR FLOW
SCONCF CFI UNIT

| | | | | p24 | |
|---|---|---|---|---|---|
| | | | p24 | [CFI- | |
| | | | [t & T] | treated] | Δp24 |
| Expt. No. | SCONCF | Virus | (ng/ml) | (ng/ml) | [% Change] |
| VAC-5 | N₂O | HIV-1Δtat-rev | 56 | 70 | +25 |
| VAC-6 | N₂O/CO₂ | HIV-1Δtat-rev | 109 | 99 | −9 |
| VAC-8 | Fr-22 | HIV-1Δtat-rev | 120 | 112 | −7 |
| VAC-9 | C₃H₈ | HIV-1Δtat-rev | 146 | 175 | +20 |
| VAC-10 | N₂O5%/ | HIV-1Δtat-rev | 107 | 82 | −23 |
| | CO₂ | | | | |
| VAC-11 | N₂ | HIV-1Δtat-rev | 107 | 143 | +34 |
| VAC-12 | CO₂ | HIV-1Δtat-rev | 14 | 15 | +7 |
| VAC-13 | Fr-23 | HIV-1Δtat-rev | 14 | 20 | +43 |

### Example 14

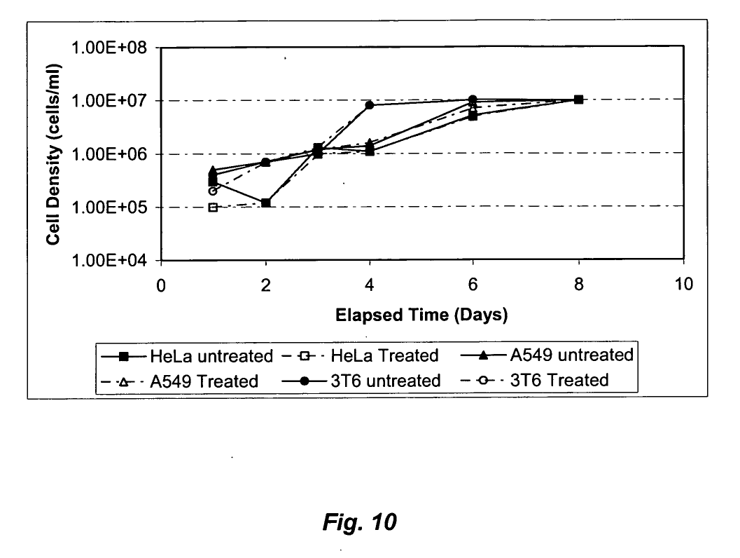

We have also demonstrated the ability of SCoNCF CFI treated fetal bovine serum, human plasma proteins such as Factor VIII and immunoglobulins, sensitive natural enzymes such as alkaline phosphatase and ac-protease inhibitor and recombinant proteins such as biosynthetic insulin to retain biochemical characteristics and biological activity. As an example of the impact of SCoNCF CFI on protein integrity and activity, several aliquots of a commercial fetal calf serum (FCS) were treated with N₂O/CO₂ at 2,000 psig and 22° C. Untreated and SCoNCF-treated FCS was compared by SMAC analysis as well as by examining the growth characteristics of several cell lines, such as A549, HeLa, 3T6, or MOPC cell lines (Table 15 and FIG. 10). SMAC analysis revealed that SCoNCF treatment had no effect on total protein, lactic dehydrogenase or alkaline phosphatase levels (SCoNCF-treated FCS was within 90% of untreated FCS; data not shown). The effect of SCoNCF-treated FCS on cell culture was determined by cytotoxicity, doubling rate as measured by manual cell counts as well as Alamar Blue staining, plating efficiency (time to confluency), and cloning efficiency. For all cell lines tested for all assays, SCoNCF-treated FCS was within 80% of untreated FCS, indicating that the SCoNCF treatment had minimal effect on the proteins, enzymes, and cytokines contained within the FCS. These results were confirmed by BioWhittaker, Walkerville, Md., using rabbit kidney cells and an MRC-5 cell line (data not shown).

TABLE 15
EFFECT OF SCONCF N2O/CO2 ON DIFFERENT CELL LINES

| | Hemocytometer | | | | | |
|---|---|---|---|---|---|---|
| | Cell Density (cells/ml) | | | | | |
| Time | HeLa | | A549 | | 3T6 | |
| (Days) | Untreated | Treated | Untreated | Treated | Untreated | Treated |
| 1 | 300000 | 100000 | 500000 | 400000 | 400000 | 200000 |
| 2 | 120000 | 120000 | 700000 | 700000 | 700000 | 700000 |
| 3 | 1300000 | 990000 | 1200000 | 1200000 | 1000000 | 1300000 |
| 4 | 1100000 | 1100000 | 1400000 | 1600000 | 8000000 | 8000000 |
| 6 | 5100000 | 4900000 | 9000000 | 7000000 | 10000000 | 10000000 |
| 8 | 10000000 | 10000000 | 10000000 | 10000000 | 10000000 | 10000000 |

### Example 15

The conditions of different experiments performed for NIBSC, London, England with parvovirus B19-spiked in human plasma samples free of B19 antibodies are recorded in Table 16. Three supercrifical fluids (Freon-22, Freon-23 and N₂O/CO₂) were used at either 25° C. or 50° C.

TABLE 16
EXPERIMENTAL CONDITIONS FOR SCONCF CFI OF HUMAN
PARVOVIRUS B19

| Experiment | | Pressure | Temperature | Flowrate | No. of |
|---|---|---|---|---|---|
| Number | SCoNCF | (bars) | (° C.) | (ml/min) | Stages |
| NIBSC-01 | Freon-22 | 206 | 50 | 4 | 2 |
| NIBSC-02 | Freon-22 | 206 | 25 | 4 | 1 |
| NIBSC-03 | Freon-23 | 206 | 50 | 4 | 1 |
| NIBSC-04 | Freon-23 | 206 | 25 | 4 | 1 |
| NIBSC-05 | N₂O/CO₂^{a} | 206/137^{b} | 50 | 4 | 2 |
| NIBSC-06 | N₂O/CO₂^{a} | 206/137^{b} | 25 | 4 | 2 |

^{a}N₂O/CO₂: N₂O with trace quantities of CO₂
^{b}206 bars in first chamber and 137 bars in the second chamber

Five or six samples were produced in each of the six experiments. A 2.5 ml aliquot of the feed was taken at the start of the treatment and stored at 4° C. during the run (named “before”) and a second 2.5 ml sample was placed at the same temperature as the SCoNCF system for the same duration as a control (named “time and temperature”).

Once the system (isobaric chamber, connecting lines, valves and gauges) was pressurized with the supercritical fluid, the sample was pumped through the isobaric chamber at the rate of 4 ml/min. After the sample had been pumped into the system, the supercritical fluid was pumped through the system at a lower flow rate (1.0 ml/min) to displace sample remaining in the system. Finally, the system was depressurized to atmospheric pressure (1.01 bars). The experimental results are listed in Tables 17 and 18.

TABLE 17
B19 DNA TITRES OF CFI-TREATED SAMPLES AND CONTROLS

B19 DNA Titre (IU/ml)

| | | | Before | Time & | CFI- |
|---|---|---|---|---|---|
| | | T | Control | Temperature | Treated |
| Expt. No. | SCoNCF | (° C.) | (4° C.) | Control | Samples |
| 01 | Freon-22 | 50 | 1 × 10¹¹ | 5 × 10¹¹ | 5 × 10¹⁰ |
| 02 | Freon-22 | 25 | 3 × 10¹¹ | 7.6 × 10¹¹ | 2 × 10¹¹* |
| 03 | Freon-23 | 50 | 2 × 10¹¹ | 1.7 × 10¹¹ | NS |
| 04 | Freon-23 | 25 | 3 × 10¹⁰ | To be | 2.5 × 10¹⁰* |
| | | | | determined | |
| 05 | N₂O/CO₂ | 50 | 1 × 10¹⁰ | 5 × 10¹⁰ | 1 × 10¹⁰* |
| 06 | N₂O/CO₂ | 25 | 2 × 10¹⁰ | 2 × 10¹⁰ | 2 × 10¹⁰* |

NS: no sample;
*volumetric average of two samples.

The B19 DNA titer remained relatively unchanged for all the samples in these experiments.

The particle: infectivity ratio for this set of experiments was very high probably due to the poor susceptibility of the cell line to B19 infection. The ratio varied from 1.3×10⁴:1 to 4.5×10⁶:1.

B19 infectivity assays of SCoNCF CFI treated samples and controls are listed in Table 18.

TABLE 18
B19 INFECTIVITY ASSAYS OF CFI-TREATED SAMPLES AND
CONTROLS

| | | | Infectious Units per ml | | |
|---|---|---|---|---|---|
| | | | Before | Time & | CFI- |
| | | | Control | Temperature | Treated |
| Expt. No. | SCoNCF | T (° C.) | (4° C.) | Control | Samples |
| 01 | Freon-22 | 50 | 1 × 10⁵ | 5 × 10^{4.5} | 5 × 10³ |
| 02 | Freon-22 | 25 | 3 × 10⁵ | 7 × 10⁴ | 2 × 10⁵* |
| 03 | Freon-23 | 50 | 2 × 10⁴ | 1.7 × 10^{4.5} | NS |
| 04 | Freon-23 | 25 | 3 × 10^{4.5} | 1 × 10⁶ | 1.7 × 10⁶* |
| 05 | N₂O/CO₂ | 50 | 1 × 10⁴ | 5 × 10^{4.5} | No |
| | | | | | detectable |
| | | | | | infectious |
| | | | | | particles* |
| 06 | N₂O/CO₂ | 25 | 2 × 10^{5.5} | 2 × 10⁵ | 1.3 × 10⁵* |

NS: no sample;
*volumetric average of two samples.

In MIBSC-01, with SCoNCF Freon-22 at 206 bars and 50° C. in a two-stage laminar flow CFI unit, there was approximately a 2log₁₀ change in infectivity titer compared with the untreated sample. The “time and temperature” control sample had a similar infectious titer to the untreated sample indicating that the loss of infectivity was due to the treatment rather that incubation of the sample at an elevated temperature.

In MIBSC-05, SCoNCF CFI inactivated more than 4log₁₀ of parvovirus B19 spiked into plasma by N₂O/CO₂ at 206 bars and 50° C. in a two-stage laminar flow CFI unit. The inactivation levels appear to be sensitive to SCoNCF type with higher levels attained with N₂O/CO₂ versus Freon-22 and Freon-23, and temperature with higher levels attained by SCoNCF at 50° C. versus 25° C. The absolute effect of temperature by itself was negligible and accounted for in time and temperature controls. It should be noted that at 25° C., the N₂O/CO₂ mixture is sub-critical whereas the mixture is supercritical at 50° C. (the critical temperatures of N₂O and CO₂ are respectively 36.41° C. and 31.1° C.). At 50° C., the N₂O/CO₂ mixture was supercritical since its pressure (206 bars) exceeded the critical pressures (respectively, 72.7 and 73.8 bars) of both N₂O and CO₂. It should be noted that the residence time is remarkably short (less than one minute) and stages (isobaric chambers) can be added to increase the level of inactivation.

Thus, preferred embodiments of the present invention have been described, which embodiments are capable of further modification and variation by those skilled in the art. Accordingly, it is intended that the examples and the description be intended for illustration purposes only and that the inventions set forth in the claims shall encompass variations and equivalents.

We claim:

1. A method for inactivating one or more virions present or potentially present in a protein-rich sample, comprising the steps of:
(a.) forming an admixture of a protein rich sample with a critical, near critical or supercritical fluid, said critical, near critical or supercritical fluid capable of being received by one or more virions associated or potentially with the protein-rich sample and upon removal of the critical, near critical, or supercritical fluids said one or more virions is inactivated, said critical, supercritical or near critical fluid comprising a mixture of carbon dioxide and nitrous oxide; and,
(b.) removing the critical, near critical or supercritical fluid to render one or more virions inactive while retaining the constituents of the virions, to form a processed protein-derived product.

2. The method of claim 1 wherein said protein rich sample has one or more proteins which proteins have an activity in said protein rich sample and following removal of said critical, near critical or supercritical fluid retain fifty percent of said activity in the processed protein derived product.

3. The method of claim 1 wherein said protein rich sample exhibits a viral activity and following removal of said critical, near critical or supercritical fluid said processed protein derived product exhibits a four log reduction in viral activity compared to said protein rich sample.

4. The method of claim 1 wherein said critical, supercritical or near critical fluid is at a temperature in the range of 0° C. to 100° C.

5. The method of claim 4 wherein said critical, supercritical or near critical fluid has a temperature that does not exceed 60° C.

6. The method of claim 1 wherein said critical, super critical or near critical fluid has a temperature range of range of 4° C. to 40° C.

7. The method of claim 1 wherein said critical, supercritical or near critical fluid mixture has a pressure in which the admixture is made and maintained which pressure is 0.75 to 20.0 times the critical pressures of nitrous oxide or carbon dioxide.

8. The method of claim 1 wherein said critical, supercritical or near critical fluid further comprises one or more fluorocarbons, alkanes, alkenes and binary gases.

9. The method of claim 1 wherein said critical, supercritical or near critical fluid is at least fifty percent nitrous oxide.

10. The method of claim 1 wherein said critical, supercritical or near critical fluid mixture further comprises one or more modifiers selected from the group consisting of ethanol, methanol, acetone, and ethylene glycol.

11. The method of claim 1 wherein critical, supercritical or near critical fluid is at approximately 10° C. to 50° C. at 800 to 3,000 psig.

12. The method of claim 1 wherein critical, supercritical or near critical fluid is a combination of nitrous oxide with trace quantities of carbon dioxide in the range from 10 to 10,000 ppm at approximately 10° C. to 50° C. at 800 to 3,000 psig.

13. An apparatus for inactivating one or more virions in a protein rich sample, comprising a vessel for forming an admixture of a protein rich sample with a critical, near critical or supercritical fluid mixture which critical, near critical or supercritical fluid mixture is capable of being received by one or more virions associated or potentially associated with said protein derived sample; upon removal of the critical, near critical, or supercritical fluid mixture one or more virions are inactivated, said super critical near critical or critical fluid comprising nitrous oxide and carbon dioxide; and depressurization means for removing the critical, near critical or supercritical fluid mixture to render one or more virions inactive while retaining the constituents of the virus to form a processed protein product.

14. The apparatus of claim 13 wherein said vessel is in communication with a continuous supply of the protein derived sample; and, said depressurization means is capable of receiving a continuous supply of the admixture of the protein derived sample and the critical, supercritical or near critical fluid.

15. A composition having viral inactivation properties comprising nitrous oxide and trace amounts of carbon dioxide in the range from 10 to 10,000 ppm at approximately 10° C. to 50° C. at 800 to 3,000 psig.
